SEQUENCE LISTING (3 sequences)

SEQ ID NO: 1 - DNA, 27 bp, Artificial
  PCR primer
caccaagaagcttttaagatttgattt

SEQ ID NO: 2 - DNA, 21 bp, Artificial
  PCR primer
ttgggatgaggaatggaaaga

SEQ ID NO: 3 - DNA, 26 bp, Artificial
  PCR primer
tggatccattctttctctcaataaca

(19) United States
(12) Patent Application Publication
(10) Pub. No.: US 2009/0013417 A1
(43) Pub. Date: Jan. 8, 2009

(54) Transgenic Animals for Assessing Drug Metabolism and Toxicity in Man

(75) Inventors: Charles Roland Wolf, Dundee (GB); Colin J. Henderson, Dundee (GB)

Correspondence Address: MYERS BIGEL SIBLEY & SAJOVEC, PO BOX 37428, RALEIGH, NC, 27627, US

(73) Assignee: IMPERIAL CANCER RESEARCH TECHNOLOGY LIMITED, London (GB)

(21) Appl. No.: 10/597,600

(22) PCT Filed: Feb. 3, 2005
(86) PCT No.: PCT/GB2005/000382

§ 371 Date: Mar. 24, 2008

(30) Foreign Application Priority Data
Feb. 3, 2004 (GB) 0402318.0

(51) Int. Cl. G01N 33/53; C12N 15/12; A01K 67/027

(52) U.S. Cl. 800/3; 435/455; 800/18

(57) ABSTRACT

A method of introducing at least one human cytochrome P450 into a non-human animal c ell in which corresponding endogenous P450 enzyme activities have been disabled, thus the method provides a way of using a non-human animal cell to make predictions regarding P450-mediated metabolism in a human. The present invention also provides transgenic non-human animals produced by the method of the invention and uses therefor, especially in assessing xenobiotic/drug metabolism and toxicity.

The present invention relates to a method of introducing at least one human cytocbrome P450 (“human P450”) into a non-human animal cell whose own equivalent endogenous cytocbrome P450 (“endogenous P450”) enzyme activities have been disabled so as to replace those endogenous P450 activities with the human equivalent. The resulting transgenic animal is referred to as a P450-humanised animal. The present invention also relates to transgenic non-human animals produced by the method of the invention and uses therefor, especially but not exclusively, the animal or cells or tissue derived therefrom is/are of use in assessing xenobiotic/drug metabolism, toxicity or other properties or functions of the human cytochrome P450-dependent monooxygenase system such as metabolism or biosynthesis of endogenous compounds.

### BACKGROUND TO THE INVENTION

A significant proportion of therapeutic drug candidates fail to become marketable drugs because of adverse metabolism or toxicity discovered during clinical trials. These failures represent a very significant waste of development expenditure and consequently there is a need for new technologies that can more reliably, quickly and economically predict at the preclinical development stage the metabolic and toxicological characteristics of drug candidates in man. At present, most pre-clinical metabolic and toxicity testing of drug candidates relies on laboratory animals, human and/or mammalian cell lines or tissues in culture. None of these methods is completely reliable in predicting metabolism or toxicity in a human subject. Metabolic data from animals can differ significantly from that obtained from a human subject due to differences in the enzymes involved. Interpretation of data from cell culture or isolated tissue studies can be problematic since such systems are unable to reflect whole body metabolism.

It is recognised that hepatic P450s are the single most important factor in determining the mammalian metabolism and toxicity of most therapeutic drugs and P450s expressed in other tissues can be critical in determining local drug metabolism, disposition or toxicity. A major technical challenge in producing P450-humanised animals lies in the large number of endogenous P450s that need to be made inactive, for example in the mouse there are 107 known P450s. It is known in the prior art to selectively inhibit some P450s with either exogenous agents or by targeted deletion of individual P450 genes. However, as many P450s involved in foreign compound metabolism exhibit overlapping substrate specificities, these approaches are not a generally effective way of anulling endogenous P450 metabolism in order to produce a transgenic animal exhibiting humanised P450 metabolism. However, all cytochrome P450s receive electrons from a single donor, cytochrome P450 reductase (CPR) and deletion of this protein would therefore inactivate all P450-mediated metabolism. While complete deletion of CPR is lethal at the embryonic stage of development, mice where the CPR gene is flanked with loxP sequences so that CPR can be conditionally deleted in the postnatal period in a specific tissue by developmentally controlled expression of cre recombinase can survive to adulthood in good health. For instance, in a co-pending application (PCT/GB03/002967 unpublished at the time of this present application) it is known to produce and use a Hepatic Reductase Null (“ERN”) mouse in which the cytochrome P450 reductase (CPR) enzyme on which all P450s depend has been deleted in the liver. IRN™ mice thus therefore completely lack P450-mediated metabolism in the liver and provide a starting point for the development of P450-humanised animals that are predictive of drug metabolism in man.

In the present invention, we have developed new strains of P450-humanised transgenic non-human animals in which endogenous P450s are made inactive in a specific tissue and one or more functional human P450s are expressed. Since liver P450s are the single most important factor in determining the metabolism and toxicity of most therapeutic drugs, it is envisaged that non-human animals humanised for P450s in the liver will be particularly useful as predictors of the metabolism and toxicity of drug candidate compounds in man.

A particular advantage of the method of the present invention resides in the production of P450-humanised transgenic animals/cells/tissues, in that the animals/cells tissues are able to combine the benefits of normal experimental animal models with those of human cell/tissue culture in a single system. This system or humanised transgenic animal will provide the pharmaceutical industry with an improved alternative for use in all pre-clinical metabolism, toxicity and drug disposition studies.

### STATEMENT OF THE INVENTION

According to a first aspect of the invention there is provided a method of introducing at least one functional human cytochrome P450 into non-human cell(s) whose own endogenous P450s have been rendered inactive, the method comprising introducing DNA encoding said at least one human P450 such that said human P450 remains functional where the cell's own endogenous P450s are inactive.

Throughout the specification and the claims which follow, unless the context requires otherwise, the word “comprise”, or variations such as “comprises” or “comprising” will be understood to imply the inclusion of a stated integer or group of integers but not the exclusion of any other integer or group of integers.

Preferably the method comprises rendering the non-human cell's own endogenous P450s inactive by deletion of the endogenous CPR gene and where function of the at least one introduced human cytochrome P450 is maintained either by it being in modified form such that it can function independently of any separate CPR protein or by introducing into the non-human cell DNA encoding a CPR such that said at least one introduced human P450 can function in the non-human animal cell(s). Reference herein to a functional human cytochrome P450 indicates that the human cytochrome P450 is enzymatically active.

In preferred embodiments of the present invention as described above and below, the transgenic non-human animal or mammal is a monkey, dog, cat, rabbit, hamster, rat, or mouse. More preferably, the transgenic non-human cell(s) are derived from a mouse.

To express active human P450 in CPR-null non-human cells, it is necessary not only to introduce human P450 genes, but also a corresponding CPR moiety to allow the expressed human P450 proteins to be enzymatically active. Expression of enzymatically active human P450(s) in a non-human animal cell whose endogenous P450s have been rendered inactive by deletion of the endogenous CPR may therefore be achieved by expressing the human P450 moiety either by; as part of a human P450-CPR fusion protein, wherein the introduced CPR moiety is tightly coupled to the P450 moiety or; in combination with a separate co-expressed human CPR.

In one embodiment of the invention therefore, a DNA sequence comprising contiguous coding sequences, with appropriate modifications, for a human P450, for example and without limitation CYP3A4 or CYP2D6, and a CPR, for example human CPR, are introduced into a non-human animal cell whose endogenous CPR gene has been deleted, so that said cell will express a human P450-CPR fusion protein in which the human P450 moiety is a fully functional P450 enzyme without the need for a separate CPR. Thus, such a fusion protein whose expression is driven by a suitable gene promoter, for example CMV or a tissue-specific promoter such as rat albumin or an inducible promoter such as CYP1A1, and introduced into transgenic mice, will provide an expression of the fusion element or protein in a constitutive or conditional fashion.

It will be appreciated that the method of conditional deletion of CPR can be directed to tissues other than the liver and can be made conditional on the administration of specific exogenous compounds by the use of different gene promoter sequences from which it is possible to drive expression of the cre recombinase. Accordingly, the method of the present invention allows for tissue selective conditional deletion of CPR in tissue other than and including the liver.

In a further embodiment of the invention, separate DNA sequences comprising, respectively, a coding sequence for a human P450, and a CPR are introduced into a non-human animal cell whose endogenous CPR gene has been deleted so that said cell will express enzymatically active human P450.

Where CPR is introduced into cells whose endogenous P450s have been made inactive by deletion of the endogenous CPR, it is preferable to express the introduced CPR in a manner that prevents reactivation of the endogenous P450s. Thus, in one embodiment of the method, in order to avoid electron transfer from the introduced CPR whether it is expressed as a separate polypeptide from the introduced human P450(s) or as part of a fusion protein, the expressed CPR protein is targeted to a specific cellular compartment where endogenous P450s are not expressed, for instance and without limitation the plasma membrane.

Preferably, the fusion protein or separate human cytochrome P450 and P450 reductase fusion proteins is/are targeted to a specific cellular component where non-human animal P540s are not expressed

Preferably, therefore, the DNA sequence introduced into a non-human animal c ell and comprising a CPR coding sequence, will also comprise a sequence chosen to direct the expressed polypeptide to an intracellular compartment remote from the endogenous P450s, for instance a sequence encoding the transmembrane spanning region of a seven transmembrane segment receptor protein.

Preferably, an intracellular targeting sequence is added to the fusion protein or separate human cytochrome P450 and P450 reductase fusion proteins

Preferably, a plurality of DNA sequences encoding different human cytochrome P450s are introduced into the non-human cell(s).

The choice of human P450 isoforms for introduction into P450-humanised non-human animals is predominantly driven by the known relative importance of various P450 isoforms in metabolism in the relevant tissue. Thus, for example, to date the single most significant isoform in the human liver CYP3A4, and so CYP3A4 is therefore the first human P450 isoform of choice for P450 humanisation of liver. The choice of human P450s for the multi-P450-humanised mouse of the present invention is dictated by the need of the user, in this respect it is expected that any one or more of the following human isoforms will be preferred 3A4, 2D6, 2C9, 1A2, 2Cl9, 2C8. However, it will be appreciated that the isoform(s) incorporated into the animal cell is/are dependent on the user's requirement. In this way, the humanised transgenic animal may be “designed” to investigate the role of specific isoforms in the metabolic process.

The present invention therefore advantageously provides a method of producing a multi-P450-humanised transgenic non-human animal.

In another embodiment of the invention, the method further includes introducing at least one further DNA sequence encoding a human protein other than a P450 that is involved in xenobiotic metabolism.

In this particular embodiment of the invention other human proteins of interest, for example and without limitation, a DNA sequence encoding a human drug transporter, for instance human Mdr-1, may be introduced into a non-human animal cell whose own corresponding endogenous gene has been deleted and in which functional human P450(s) are also expressed where the endogenous P450s have been made non-functional will permit the functions of the, for example, human drug transporter protein to be studied in the non-human animal in the context of human P450 metabolism.

It will be appreciated that this aspect of the invention includes any one or more of the features as hereinbefore described.

According to a yet further aspect of the invention there is provided use of human cells, especially hepatocytes, introduced into an immune-deprived reductase null animal so as to investigate contribution of human cells or hepatocytes in P450-mediated product metabolism and/or toxicity and/or drug candidate screening.

In this embodiment of the invention, human cells or hepatocytes may grow in, for example the liver or spleen of SCID (severe combined immune deficiency) mice. SCID mice are homozygous for the Prkdc^{scid} mutation and lack both T and B cells due to a defect in V(D)J recombination. Therefore, these mice easily accept foreign tissue transplants, including human tumours, making them effective models for testing new cancer treatments and as hosts for human immune system tissues (i.e., SCID-hu).

Accordingly, in this embodiment of the invention only the human cells or hepatocytes will contribute to cell or hepatocyte-mediated P450 metabolism and thus may be advantageously used as a human metabolism model in the absence of background mouse metabolism.

According to a yet further aspect of the invention there is provided use of a transgenic animal, tissues and/or cells derived therefrom as hereinbefore described that have been modified to contain and express DNA encoding at least one human P450 and/or another protein involved in metabolism so as to investigate human P450 mediated metabolism in said a transgenic animal, tissues and/or cells derived therefrom.

The present invention advantageously provides a tool for the investigation not only of metabolism of xenobiotics but of the functions of human P450s in the metabolism and biosynthesis of endogenous compounds such as and without limitation neurosteroids, and in disease states such as and without limitation, choleastasis, artherogenesis, hormonal imbalances, neurological disorders, degenerative diseases, skin conditions, cardiovascular disease, cancer and glaucoma and any other disease in which P450s play a role.

A yet further advantage of the present invention is in the provision of, for example, a CYP3A4-humanised transgenic mouse which may be used for problem-solving studies in drug metabolism and toxicity.

The invention will now be described by way of example only with reference to the following figures wherein:

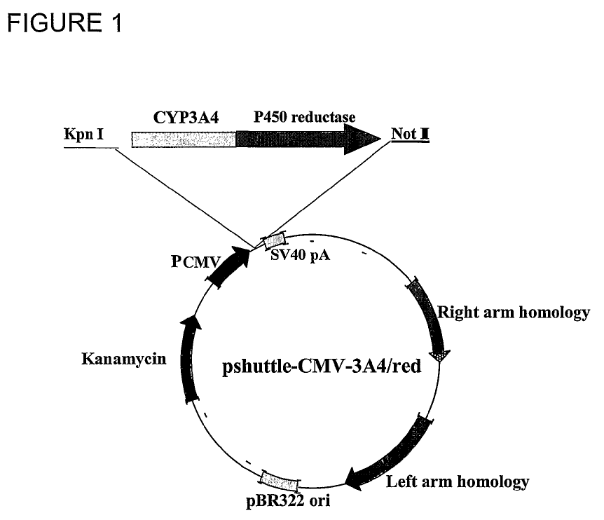

FIG. 1 illustrates the pshuttle-CMV-3A4, fall length of human CYP3A4 cDNA fusion with human P450 reductase cDNA (codons for first 57 amino acids deleted) ligated into the Kpn I/Not I site of the shuttle vector pshuttle-CMV;

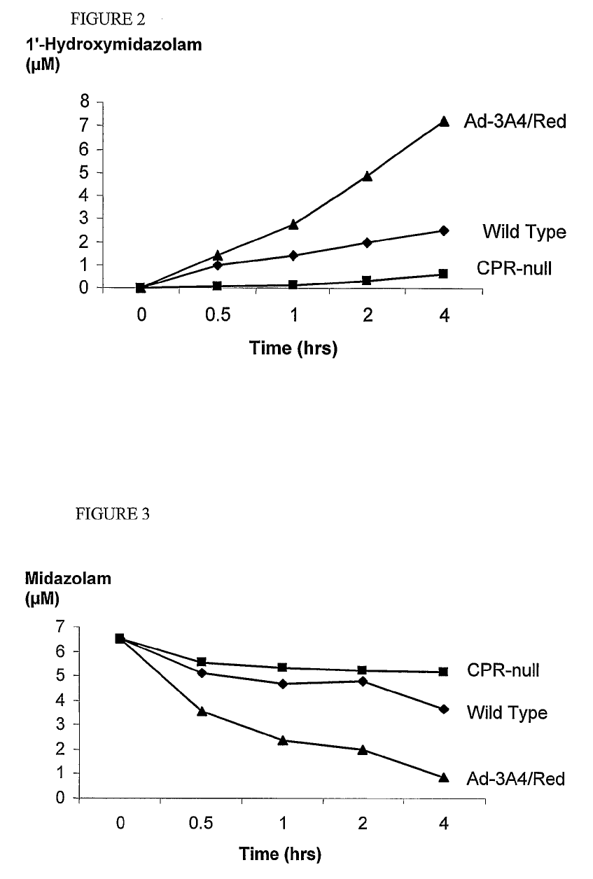

FIG. 2 illustrates 1′ hydroxy-midazolam production by mouse hepatocytes during midazolam metabolism; and

FIG. 3 illustrates midazolam concentrations during midazolam metabolism by mouse hepatocytes.

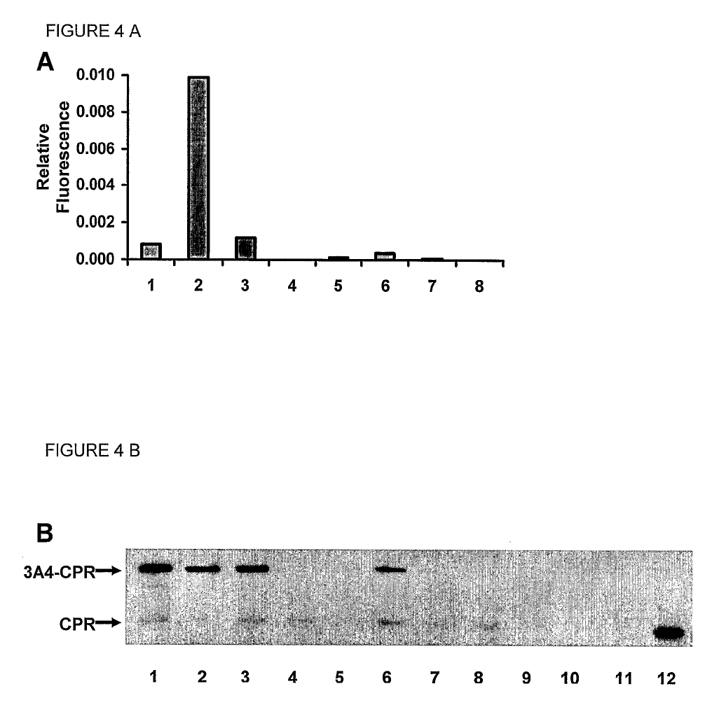

FIG. 4A shows a bar chart of relative fluorescence determined by a RT-PCR assay of CYP34A-CPR fusion construct mRNA expression in Ad-3A4/red virus-infected mouse liver.

FIG. 4B shows an immunoblot analysis of CYP34A-CPR fusion protein expression in Ad-3A4/red virus-infected mouse liver.

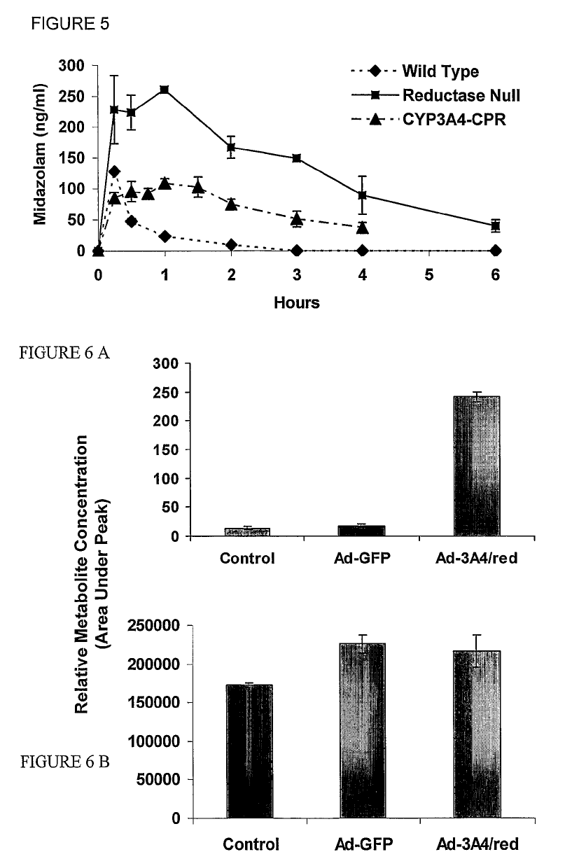

FIG. 5 shows a graph of blood levels of midazolam (ng/ml) in HRN mice infected with Ad-3A4/red virus.

FIG. 6 shows a bar chart of relative metabolite concentration (area under peak) form control, Ad-GFP and AD-3A4/red infected cells; FIG. 6A is metabolism of midazolam and FIG. 6B is of bufuralol.

### DETAILED DESCRIPTION OF THE INVENTION

### Materials and Methods

### The HRN™ Mouse

All P450s require reducing electrons supplied by the enzyme cytochrome P450 reductase (CPR). Deletion of CPR therefore simultaneously inactivates all P450s. While CPR deletion is lethal in the embryo, HRN™ mice use a developmentally regulated conditional CPR deletion targeted to post-natal liver cells. HRN™ ice therefore survive to adulthood and can breed while nevertheless completely lacking hepatic P450-mediated metabolism (Henderson CJ et al. J Biol Chem. 278:13480-6, 2003). They therefore provide a suitable background on which to express human P450 activities in order to achieve P450 humanisation.

### Transgenic Mouse Production

An adenoviral vector may be used to introduce the human P450/CPR combination to HRN™ cells. Alternatively, germ line transgenic animals incorporating the same transgenes can be produced. This is achieved by first generating transgenic mice incorporating the selected CYP3A4/CPR humanisation transgenes and then crossbreeding these with HRN™ mice to produce CYP3A4-humanised animals. Production of CYP3A4/CPR transgenic mice is achieved by using targeted transfection of embryonic stem cells and subsequent blastocyst injection. Crossbreeding of CYP3A4/CPR transgenics with HRN™ to produce P450-humanised animals may be used for the production of multi-P450-humanised mice. Alternatively, embryonic stem cells may be produced where the CPR gene is flanked by loxP sites and where expression sequences for targeted human P450(s) and CPR or for human P450-CPR fusion protein(s) have been introduced. Animals derived from such embryonic stem cells may then be crossbred with various animal strains in which cre recombinase is expressed under the control of different promoters to produce offspring P450-humanised in different tissues or under different induction conditions, depending on the tissue specificity or inducibility of the promoter controlling cre recombinase expression.

### Humanisation Strategies

In order to establish the optimal method of expressing functional human P450 activities in HRN™ cells, experimental transgenes encoding cytosolic fusion proteins, targeted fusion proteins, targeted CPR with separate cytosolic P450s are compared and evaluated. In each case, the ability of the P450 to interact with the CPR component is determined by expressing these alternatives in appropriate cell culture systems and then testing them in vivo by adenovirus transfection of HRN™ mice.

### Cell Line for Evaluation of Human P450 Transgenes

We use a suitable immortalised cell line for initial in vitro evaluation of the various transgene strategies. This is based on an immortalised cell line derived from HRN™ or on an existing cell line known to be deficient in CPR and P450 expression (e.g. CHO cells), which is then transfected to over express recombinant mouse P450. The cell line will is then used to test experimental transgenes for their ability to produce human P450 activity without activation of the mouse P450.

### Adenoviral Delivery of Transgenes In Vivo

The invention requires transgene expression in the liver and adenovirus transgene delivery to liver cells of mature mice is a feasible method of introducing human P450 transgenes for appropriate expression in HRN™. Previous published work has shown that adenoviral vectors can deliver transgenes to virtually all hepatocytes in mouse liver. We used this method to initially evaluate the suitability of the various human P450 expression strategies outlined above in vivo. This approach permits the generation of prototype humanised animals for evaluation relatively quickly and more cost effectively. Once generated, adenovirus-transfected animals are dosed with appropriate test compounds to evaluate metabolism as having the correct characteristics for metabolism by the introduced human P 450 without evidence of endogenous P450 reactivation.

### In Vivo Validation of Transgenic Mice

Information relating to drug bioavailability, toxicology and active transport data is collected. Such data shows humanised mice acting as effective predictors of the properties of a given compound in man where wild-type mice do not. Data is obtained from humanised HRN™ mice and control HRN™ mice. Test compounds with known P450 metabolic profiles in mouse and man are selected and administered orally to humanised and control mice. Mass spectroscopic assays for each drug has been developed and used to determine circulating concentrations of administered drug and known metabolites. Where appropriate, pathological investigations may be conducted to establish compound toxicity.

### Isolation of Mouse Hepatocytes

Wild-type and hepatic CPR-null mice were anaesthetised by intraperitoneal injection of pentobarbitone (100 mg/kg body weight) and the livers immediately perfused with Calcium-free Krebs-Ringer phosphate buffer, pH 7.4 (KRPB) via the hepatic portal vein at a flow rate of 12 ml/min for 10 minutes followed by Calcium-free Krebs-Ringer hydrogen carbonate buffer, pH 7.4 (KRHB) at 12 ml/min for 5 minutes and then 0.2 mg/ml collagenase in KRHB containing 0.3 mM calcium chloride at 12 ml/min for 10 minutes. Cell suspensions were then produced by mechanically disrupting removed liver tissue, filtration through bolting cloth and washing in KRHB.

Mouse hepatocytes were grown at 37° C. in complete Leibowitz L15 (CL-15) medium supplemented with 8.3% fetal calf serum (FCS), penicillin (41 IU/ml), streptomycin (41 μg/ml), tryptose phosphate broth (8.3%, v/v), hydrocortisone 21-hemisuccinate (10 μM), insulin (1 μM), hemin (5 μg/ml) and glutamine (240 μg/ml). Hepatocytes were seeded at a density of 8×10⁵ cells per well on 6-well plates and incubated at 37° C. for one day before infection with human cytochrome P450 3A4-human cytochrome P450 reductase fusion gene by adenoviral vector.

### Ad-3A4/Red Plasmid Construction and Virus Preparation

The human P450 3A4 (CYP3A4)-P450 reductase (CPR) fusion cDNA comprised contiguous modified CYP3A4 and CPR coding sequences. The CYP3A4 sequence was modified at the N-ternminus by insertion of a Kpn I restriction site following the Kozak sequence and just before ATG start codon and by deletion of the TGA stop codon. The CPR sequence was modified by deletion of coding sequence for the first 57 amino acids (ER anchor) and flanked at the C-terminus by a NotI restriction site. The CYP3A4-CPR fusion cDNA was inserted into the shuttle vector pshuttle-CMV (Stratagene) [FIG. 1] and Ad-3A4/red virus prepared using the AdEasy XL Adenoviral vector system (Stratagene).

### Virus Infection and Determination of Catalytic Activity in Cultured Cells

Hepatocytes from HRN™ mice were infected with Ad-3A4/red adenovirus according to methods of the AdEasy XL Adenoviral vector system (Stratagene). After 4-days of infection with virus, the cell medium was replaced with 2 ml of fresh CL-15 plus bufaralol (10 μM) or midazolam (10 EM). After 0.5, 1, 2 and 4 hours of incubation, 200 gi aliquots of medium were transferred to Eppendorf tubes with 200 μl ice-cold acetonitrile containing 5 μM dextrorphan as internal standard for mass spectrometric analysis.

### Mass Spectrometric Analysis for Bufuralol, Midazolam and Specific Metabolites

Analysis for the loss of bufuralol and midazolam was carried out using reverse-phase HPLC with tandem mass spectrometric detection (LC-MS/MS). An aliquot of each standard and sample (20 μL) was injected onto the liquid chromatography system and eluted under isocratic conditions of 50% H₂O with 0.1% (v/v) Formic acid and 50% Acetonitrile at a flow rate of 0.5 mL/min through a Phenomenex MercuryMS Luna 3 μM C18(2), 20×2 mm HPLC column.

Positive ions for parent compounds and specific fragment products were monitored in Multiple Reaction Monitoring (MRM) mode using a Micromass Quaffro Micro Mass Spectrometer with Micromass MassLynx software version 3.5, monitoring for the positive bufuralol parent ion at 262.41 m/z with the specific product ion at 188.12 m/z; for the positive midazolam parent ion at 326.35 m/z with the specific product ion at 291.22 m/z and for internal standard dextrorphan parent ion at 258.47 m/z with the specific product ion at 157.21 m/z.

Analysis for the appearance of the specific metabolites 1′-hydroxybufuralol and 1′-hydroxymidazolam was carried out as above, but using gradient elution from the HPLC column and MRM of the positive 1′-hydroxybufuralol parent ion at 278.4 m/z with the specific product ion at 186.2 m/z and of the positive 1′-hydroxymidazolam parent ion at 342.3 m/z with the specific product ion at 203.2 m/z.

Real-Time Quarztitative Polymerase Chain Reaction (RT-PCR) Assay of Ad-3A4/Red mRNA

Messenger RNA was prepared from liver tissue and assayed for the presence of CYP3A4-coding sequences by real-time quantitative PCR. RNA was diluted to 0.1 μg/μl and DNase-treated (Promega UK) at 37° C. for 10 mins. cDNA was synthesized using 100 units of Superscript II reverse transcriptase (Invitrogen) and 0.15 g of random primers (Promega UK) in a solution of 50 mM Tris/HCL, pH 8.3, 75 mM KCL and 3Mm MgCl₂ containing 10 mM dithiothreitol and 1 mM dNTPs. The reaction was equilibrated at 25° C. for 10 mins before synthesis proceeded at 42° C. for 50 min. The reaction was terminated by incubation at 70° C. for 10 min, and cDNA was diluted to 200 μl and stored at −20° C. until use. Matching oligonucleotide primers and probes for real-time PCR were designed using Primer Express™ (PerkinElmer Applied Biosystems) software. Each PCR mix (12.5 μl) contained 2.5 μl cDNA, 299 nM forward primer (5′-CACCAAGAAGCTTTTAAGATTTGATTT-3′) (SEQ ID NO:1) and reverse primer (5′-TTGGGATGAGGAATGGAAAGA-3′) (SEQ ID NO:2) and 1001 nM probe (5′-TGGATCCATTCTTTCTCTCAATAACA-3′) (SEQ ID NO:3) in 1× (final concentration) TaqMan® Master Mix (PerkinElmer). Amplification of cDNA was performed over 41 cycles in the Prism Model 7700 Sequence Detector instrument. The first cycle was performed using 50° C. for 2 min, followed by 95° C. for 10 min. Cycles 2-41 were performed at 95° C. for 15s, followed by 60° C. for 1 min. Reactions were monitored by measuring fluorescence at 518 nm with excitation at 494 nm. Each assay was performed in triplicate, and specificity of PCR reactions from various primers were examined routinely by agarose-gel electrophoresis. GAPDH was used as an internal standard and results were analysed using 7700 system software.

### Immunoblot Analysis of Ad-3A4/Red Expression

Hepatic microsome proteins, prepared from snap-frozen tissues, were loaded at 5 mg protein per lane onto 9% polyacrylamide gels and separated by electrophoresis in buffer containing 10% sodium dodecyl sulphate (SDS). Separated proteins were transferred to nitrocellulose membranes and immunostained with a polyclonal antiserum to human cytochrome P450 reductase.

### EXAMPLE 1

### Generalised Scheme for P450 Humanisation in a Tissue-Specific or Inducible Manner

A transgenic mouse is generated carrying the following transgenes: (1) a human P450-CPR fusion gene with intracellular targeting sequences is preceded by an interfering gene fragment flanked by loxP sequences preceded by a ubiquitous promoter, for instance the ROSA26 promoter where the interfering fragment is designed so that no transcription of the fusion gene occurs; (2) the endogenous CPR gene replaced by a CPR gene flanked by loxP sequences; (3) a cre recombinase gene under the control of a tissue-specific or inducible promoter. Under conditions where ere recombinase is expressed, either as the result of cellular differentiation or of administration of inducing agents, recombination at the loxP sites results in deletion of the endogenous CPR and consequent deactivation of endogenous P450s together with activation of expression of the human P450-CPR fusion gene by virtue of deletion of the interfering fragment. In this way, both deactivation of endogenous P450s and expression of the functioning human P450(s) are restricted to a specific tissue type or to specific conditions of induction.

### EXAMPLE 2

### Expression of Human Cytochrome P450 3A4 Activity in Isolated Hepatocytes from Hepatic CPR-null Mice

In this study, isolated hepatocytes from hepatic CPR-null mice were infected with adenovirus carrying a human cytochrome P450 3A4-cytochrome P450 reductase fusion gene under control of the CMV promoter. This resulted in expression of human P450 3A4 activity in the infected hepatocytes showing that cells from the CPR-null animals can be used as the basis of ‘humanised’ metabolic systems.

In order to determine the effect of human recombinant CYP3A4/reductase on hepatic CPR-null hepatocytes P450 monooxygenase activities, the catalytic activity of the CYP3A4 was measured using midazolam, monitoring disappearance of the added drug and accumulation of the metabolites 1′hydroxy-midazolam in hepatocytes from wild-type mice and hepatocytes from hepatic CPR-null mice with or without infection with the Ad-3A4/red fusion vector.

After 4 hours incubation, 1′hydroxy-midazolam concentrations in hepatocyte cultures from hepatic CPR-null mice incubated with midazolam were only approximately 25% of those produced in cultures from wild-type mice. However, cultures from CPR-null mice that had been infected with Ad-3A4/red produced approximately 3 times as much 1′hydroxy-midazolam as those from wild-type animals as can be seen in the graph represented by FIG. 2, which shows 1′hydroxy-midazolam production during midazolam metabolism by mouse hepatocytes over a 4 hr incubation period.

Corresponding effects were seen on the disappearance of midazolam as is apparent from FIG. 3 (midazolam concentration during midazolam metabolism by mouse hepatocytes over a 4 hr incubation period).

### EXAMPLE 3

### Expression of the CYP3A4-CPR Fusion Protein in HRN Mice

Eight HRN mice received Ad-3A4/red virus (100 μl at 10⁹ pfu/ml intravenously). The animals were killed four days later and liver tissue collected and snap-frozen in liquid nitrogen.

RT-PCR analysis revealed expression of CYP3A4-CPR mRNA in livers of four of the eight infected mice, as indicated by the presence of CYP3A4 coding sequence. FIG. 4A shows CYP3A4-CPR fusion construct mRNA expression in Ad-3A4/red virus-infected mouse liver determined by RT-PCR assay of CYP3A4 coding sequence.

With regard to FIG. 4B, we have shown CYP3A4-CPR fusion protein expression in Ad-3A4/red virus-infected mouse livers determined by immunoblot analysis of hepatic microsome proteins with an anti-CPR antiserum. Arrows indicate bands due to the CYP3A4-CPR fusion protein (3A4-CPR) in adenovirus-infected HRN mice and of native cytochrome P450 reductase (CPR) in wild-type mice. Lanes are identified as follows: 1-8=Ad-3A4/red virus-infected HRN mice 1-8 respectively; 9,10=Blank; 11=uninfected HRN mouse liver microsomes (no adenovirus); 12=Wild-type mouse liver microsomes. Thus, expression of the fusion protein was confirmed by immunoblotting of hepatic microsomal proteins with the anti-CPR antiserum which revealed reactive bands in microsomes from the same four mice that had yielded positive results for the mRNA. These bands ran more slowly than that produced by the native CPR protein, consistent with the higher molecular mass of the fusion protein (FIG. 4B).

These results demonstrate that adenoviral delivery of the Ad-3A4/red plasmid in vivo results in expression of the CYP3A4-CPR fusion protein in liver.

### EXAMPLE 4

### Human CYP3A4 Metabolism in Ad-3A4/red Virus-Infected HRN Mouse Liver

Four adult female HRN mice received 100 μl of 10⁹ pfu/ml Ad-3A4/red virus intravenously. Seventy-two hours later the animals received oral doses of 2.5 mg/kg midazolam. Blood samples were collected from each animal at 15, 30, 45, 60, 90, 120, 180 and 240 minutes after midazolam dosing. Midazolam concentrations were assayed by mass spectroscopy.

FIG. 5 shows blood midazolam concentrations following oral administration of 2.5 mg/kg midazolam. Values are mean±standard error of the mean in HRN animals infected with the Ad-3A4/red virus compared to concentrations in non-infected HRN animals and with wild-type animals. As expected, midazolam was rapidly metabolized by the wild-type animals. Circulating midazolam concentrations reached a peak value of 128 ng/ml and had become undetectable again within 3 hours. In comparison, HRN animals exhibited severely impaired midazolam metabolism with circulating midazolam concentrations reaching a peak value of 260 ng/ml and remaining detectable beyond 6 hours. In HRN animals infected with the Ad-3A4/red virus, however, there was clear evidence of restored ability to metabolize midazolam with peak midazolam concentration achieved being only 109 ng/ml.

These results demonstrate that adenoviral delivery of the Ad-3A4/red plasmid and expression of the CYP3A4-CPR fusion protein in HRN mice restores the animal's ability to metabolize midazolam, a substrate of CYP3A4.

### EXAMPLE 5

### Lack of Electron Transfer Between the CYP3A4-CPR Fusion Protein and Cytochrome P450s

Expression of a cytochrome P450-CPR fusion protein in HRN mouse cells introduces the possibility of electron transfer from the introduced CPR moiety reactivating endogenous cellular cytochrome P450s. It is preferable from the point of view of producing a humanised cell if such ‘crosstalk’ between the fusion protein CPR moiety and cellular cytochrome P450s is kept to a minimum. To establish whether such crosstalk takes place, expression of the CYP3A4-CPR fusion protein was induced in two cell lines by infection with the Ad-3A4/red adenoviral vector.

With regard to FIG. 6, Chinese Hamster Ovary (CHO) cell line transfected with human cytochrome P450 2D6 (CYP2D6) grown in culture were infected with Ad-3A4/red. Four days later, fresh medium containing either midazolam or bufaralol at 10 μM was added to the cultures and four hours later medium was collected for analysis by mass spectroscopy for the presence of 1′-hydroxymidazolam or 1′-hydroxybufuralol respectively. A bar chart was made of relative metabolite concentration values shown are relative ‘area under the peak’ values. Each value is mean±standard error for four culture wells. ‘Control’ indicates uninfected CHO/2D6 cells; ‘Ad-GFP’ indicates c ells infected with a control adenovirus containing green fluorescent protein cDNA; ‘Ad-3A4/red’ indicates cells infected with the Ad-3A4/red CYP3A4-CPR fusion protein adenovirus.

In CHO cells expressing CYP2D6, whether uninfected or infected with a control adenovirus vector introducing a green fluorescent protein (Ad-GFP) mRNA, midazolam metabolism was extremely slow. Infection with Ad-3A4/red increased midazolam metabolism by more than 10-fold (FIG. 6A). In contrast, metabolism of the CYP2D6 substrate bufuralol was substantial in control CHO/2D6 cells and hardly altered at all after infection with Ad-3A4/red (FIG. 6B).

These results demonstrate that while expression of the CYP3A4-CPR fusion protein results in markedly increased metabolism of midazolam which is predominantly a CYP3A4 substrate, metabolism of bufuralol which is predominantly a CYP2D6 substrate is unaffected. These results indicate that while the CPR moiety of the CYP3A4-CPR fusion protein is efficient at transferring electrons to the CYP3A4 moiety of the fusion protein, it does not significantly interact with the separate CYP2D6 protein. The fusion protein CPR is not therefore likely to cause significant reactivation of and cellular cytochrome P450s in cells from the HRN mouse or other CPR-null cells.

We have been able to demonstrate the principle of the humanisation using P450-CPR fusion genes: cultured hepatocytes from HRN™ mice have been infected with an adenovirus carrying an inserted artificial gene encoding a human CYP3A4-CPR fusion protein. The fusion protein comprising a human CYP3A4 enzymatic profile but is able to be independent of any separate cellular CPR since its own CPR moiety supplies the reducing electrons required by the P450 moiety. CPR-null hepatocytes infected with the fusion protein gene have been compared with uninfected CPR-null hepatocytes and hepatocytes from normal mice in their ability to metabolise test compounds and are known to have differing susceptibility to metabolism by human CYP3A4. Hepatocytes from CPR-null mice had very much reduced ability to metabolise the test compounds compared to normal hepatocytes. Infection with the human CYP3A4-CPR fusion gene restored metabolism, more strongly for the compound known to be a good substrate for human 3A4 than for compound known to be a poor substrate. These results demonstrate that human CYP3A4 activity could be expressed over the mouse P450-null background and illustrates the basic concept of 450 humanisation.

1. A method of introducing at least one functional exogenous human cytochrome P450 into a non-human cell having inactive endogenous cytochrome P450s, the method comprising introducing DNA encoding said at least one exogenous human cytochrome P450 to provide at least one functional human cytochrome P450 whereas the endogenous cytochrome P450s are inactive.

2. The method according to claim 1 wherein an endogenous cytochrome P450 reductase (CPR) gene is deleted from the genome of the non-human cell and wherein the function of the at least one exogenous human cytochrome P450 is maintained by modifying the exogenous human cytochrome P450 to function independently of a separate CPR moiety or introducing into the non-human cell a DNA encoding a CPR gene to provide an exogenous CPR moiety to interact with the exogenous human cytochrome P450.

3. The method according to claim 1 wherein the non-human cell is derived from a monkey, dog, cat, rabbit, hamster, rat, or mouse.

4. The method according to claim 3 wherein the non-human cell is derived from a mouse.

5. The method according to claim 1 wherein a plurality of DNA sequences encoding different human cytochrome P540s are introduced into the non-human cell.

6. The method according to claim 1 wherein the human cytochrome P450 is selected from the group comprising 3A4, 2D6, 2C9, 1A2, 2C19 and 2C8

7. The method according to claim 1 wherein enzymatically active human cytochrome P450 enzymes are expressed through expression of a fusion protein comprising a cytochrome P450 moiety and a cytochrome P450 reductase protein or co-expression of a separate human cytochrome P450 moiety and a separate cytochrome P450 reductase protein.

8. The method according to claim 7 wherein expression of the human cytochrome P450 is driven by a gene promoter.

9. The method according to claim 8 wherein the promoter is CMV, a tissue-specific rat albumin promoter or CYP1A1.

10. The method according to claim 7 wherein expression of the fusion protein or co-expression of the separate human cytochrome P450 moiety and the P450 reductase protein is constitutive or conditional.

11. The method according to claim 7 wherein the fusion protein or co-expression of the separate human cytochrome P450 moiety and the P450 reductase protein is targeted to a specific cellular component where non-human animal P540s are not expressed.

12. The method according to claim 11 wherein an intracellular targeting sequence is added to the fusion protein or co-expressed with the separate human cytochrome P450 moiety and the P450 reductase protein.

13. The method according to claim 1 further including the step of introducing into a nonhuman cell at least one DNA sequence encoding a human protein involved in xenobiotic metabolism other than a cytochrome P450.

14. The method according to claim 13 wherein the at least one DNA sequence encoding the human protein encodes a drug transporter protein.

15. The method according to claim 14 wherein the DNA sequence encoding the human protein encodes Mdr.

16. A method of assessing human cytochrome P450-mediated metabolism, comprising using a transgenic animal, tissues and/or or cells comprising a DNA encoding at least one human P450 and/or another protein involved in metabolism.

17. The method according to claim 16 wherein results of the assessment of human cytochrome P450-mediated metabolism correlates to assessment of disease states selected from the group consisting of choleastasis, artherogenesis, hormonal imbalances, neurological disorders, degenerative diseases, skin conditions, cardiovascular disease, cancer and glaucoma and any other disease in which P450s play a role.

18. A method of using human cells introduced into an immune-deprived reductase null animal so as to investigate contribution of said human cells in P450-mediated product metabolism and/or toxicity and/or drug candidate screening.

19. The method according to claim 18 wherein said human cells are hepatocytes.

20. A CYP3A4/CPR transgenic HRN™ mouse.

21. Use of a mouse according to claim 20 in pre-clinical and toxicity studies.
